(19) United States
(12) Patent Application Publication
(10) Pub. No.: US 2011/0172171 A1
(43) Pub. Date: Jul. 14, 2011

(54) TAURINE OR TAURINE-LIKE SUBSTANCES FOR THE PREVENTION OF BRAIN OEDEMA

(76) Inventors: Serge Picaud, Paris (FR); José-Alain Sahel, Paris (FR); Firas Jammoul, Paris (FR)

(21) Appl. No.: 13/063,552

(22) PCT Filed: Sep. 4, 2009
(86) PCT No.: PCT/EP2009/061464

§ 371 Date: Mar. 11, 2011

(30) Foreign Application Priority Data
Sep. 12, 2008 (EP) 08305548.3

(51) Int. Cl. A61K 38/06; A61K 31/185; A61K 31/198; A61K 31/7076; A61K 31/575; A61K 31/549; A61K 31/4462; A61K 31/662; A61K 31/47; A61K 31/495; A61K 31/675; A61K 31/714; A61K 31/519; A61K 31/525; A61K 31/455; A61K 31/51; A61K 31/197; A61K 31/5513; A61K 31/515; A61K 31/4015; A61K 31/53; A61K 31/4166; A61K 31/357; A61K 31/4535; A61P 25/08; A61P 7/10

(52) U.S. Cl. 514/21.9; 514/578; 514/562; 514/46; 514/561; 514/182; 514/222.5; 514/315; 514/114; 514/311; 514/252.12; 514/89; 514/52; 514/249; 514/251; 514/356; 514/276; 514/563; 514/221; 514/271; 514/425; 514/242; 514/391; 514/454; 514/326

(57) ABSTRACT

The present invention relates to taurine or taurine-like substances for the prevention of brain oedema, particularly brain intramyelinic oedema and more particularly brain intramyelinic oedema induced by an anti-convulsive drug such as vigabatrin. The invention relates to a substance selected from the group consisting of taurine, a taurine precursor, a taurine metabolite, a taurine derivative, a taurine analog and a substance required for the taurine biosynthesis for the prevention of brain oedema.

### FIELD OF THE INVENTION

The prevention invention relates to taurine or taurine-like substances for the prevention of brain oedema, particularly brain intramyelinic oedema and more particularly brain intramyelinic oedema induced by an anti-convulsive drug such as vigabatrin.

### BACKGROUND OF THE INVENTION

Brain oedema may be observed in various pathological conditions including hypertensive encephalopathy, cerebral malaria, lesions of the anterior interosseous nerve, lateral amyotrophic sclerosis, Lyme disease, hepatic encephalopathy, Wernicke's encephalopathy, traumatic brain injury, stroke, intracerebral haemorrhage, intracranial hypertension. Brain oedema can also occur as a serious side effect of anti-convulsive drugs (e.g. anti-epileptic drugs) (Chason et al., 1996; Kim et al., 1999; Polster et al., 2001; Anneken et al., 2008, Wheless JW en Al., 2009; Pearl PL, et Al., 2009 and Milh M, et Al., 2009).

For example, vigabatrin, an anticonvulsant drug marketed almost all over the world, has been shown to induce lesions in the splenium of the corpus callo sum of a few vigabatrin- treated patients (Kim et al., 1999). MRI abnormalities indicative of brain intramyelinic oedema were very recently reported in children treated with vigabatrin for infantile spasms (Wheless JW en Al., 2009; Pearl PL, et Al., 2009 and Milh M, et Al., 2009). These lesions are considered to be reversible demyelination related to the vigabatrin treatment. However, no microvacuolation and gliosis had been detected on post-mortem histopathological sections (Cannon et al., 1991; Hammond et al., 1992). In animals, vigabatrin was first shown to cause white matter vacuolation with intramyelinic oedema in rats, mice and dogs (Butler et al., 1987; Gibson et al., 1990; Schroeder et al., 1992; Yarrington et al., 1993; Preece et al., 2004). These lesions were also detected by MRI imaging in the cerebellar white matter, cerebral cortex, thalamus and hippocampus in rats and dogs (Jackson et al., 1994; Peyster et al., 1995; Preece et al., 2004). As in human patients, these MRI studies indicated an increase in T2 relaxation time measurements.

Thus, there is a need in the art for substances that would allow preventing brain oedema, particularly brain intramyelinic oedema. Particularly, brain oedema induced by an anti-convulsive drug such as vigabatrin provides a model for testing molecules preventing the development of brain oedema.

### SUMMARY OF THE INVENTION:

The present invention relates to a substance selected from the group consisting of taurine, a taurine precursor, a taurine metabolite, a taurine derivative, a taurine analog and a substance required for the taurine biosynthesis for the prevention of brain oedema.

The present invention also relates to a pharmaceutical composition for the prevention of brain oedema which comprises a substance as above described and optionally one or more pharmaceutically acceptable excipients.

Finally, the present invention relates to a pharmaceutical composition for the treatment of convulsive disorders which comprises (i) a first active ingredient which is an anti-convulsive drug; and (ii) a second active ingredient selected from the group consisting of taurine, a taurine precursor, a taurine metabolite, a taurine derivative, a taurine analog and a substance required for the taurine biosynthesis and (iii) optionally one or more pharmaceutically acceptable excipients.

### DETAILED DESCRIPTION OF THE INVENTION:

### Therapeutic methods and uses

Surprisingly, the inventors demonstrate that a supplementation of vigabatrin-treated individuals with taurine prevents brain oedema induced by vigabatrin.

Thus, an object of the present invention relates to a substance selected from the group consisting of taurine, a taurine precursor, a taurine metabolite, a taurine derivative, a taurine analog and a substance required for the taurine biosynthesis, for the prevention of brain oedema.

In a particular embodiment of the invention, the brain oedema is a brain intramyelinic oedema.

As used herein, the term “brain oedema” denotes an excess accumulation of water and sodium in the intracellular and/or extracellular spaces of the brain.

As used herein, the term “brain intramyelinic oedema” denotes vacuolation of the white matter. This vacuolation is indicated by vacuole formation in the white matter on histological sections. However, it can also be diagnosed by different in vivo imaging techniques. For instance, brain MRI could detect such brain alterations in different brain structures such as the splenium of the corpus callosum, the hippocampus, the thalamus, the cerbellar white matter, the cortex by reduced T1 and/or increased T2 signal intensities. Examples of brain oedema are not limited to those observed following treatment with anti-convulsive drugs, they also include brain oedema described in hypertensive encephalopathy, in cerebral malaria, in lesions of the anterior interosseous nerve, in lateral amyotrophic sclerosis, in Lyme disease, in hepatic encephalopathy, in Wernicke's encephalopathy, in traumatic brain injury, in stroke, in intracerebral haemorrhage, in intracranial hypertension.

As used herein, the term “taurine” refers to 2-aminoethanesulfonic acid.

As used herein, “taurine precursors” encompass substances that, when they are administered to a human or an animal, can be transformed, directly or indirectly, into taurine.

As used herein, “taurine metabolites” encompass substances that are produced in vivo by transformation of taurine.

As used herein, “taurine derivatives” encompass substances that are structurally close to taurine but possess at least one structural difference, such as one or more chemical changes, e.g. at least one replacement of an atom or a chemical group found in taurine by a distinct atom or a distinct chemical group.

As used herein, “taurine analogs” encompass substances that are chemically distinct from taurine but which exert the same biological activity.

As used herein, “substances required for taurine biosynthesis” encompass all substances that are involved in the in vivo taurine biosynthesis including enzymes and enzyme cofactors, thus including cysteine dioxygenase (EC 1.13.11), sulfinoalanine decarboxylase (EC 4.1.1.29) and cofactors thereof.

As intended herein, taurine precursors, taurine metabolites, taurine derivatives, taurine analogs and substances required for the taurine biosynthesis may be collectively termed “taurine-like substances”.

In a particular embodiment, taurine precursors are selected from the group consisting of cysteine, cystathionine, homocysteine, S-adenosylhomocysteine, serine, N-acetyl-cysteine, glutathione, N-formylmethionine, S-adenosylmethionine, betaine and methionine. In another particular embodiment, taurine metabolites are selected from the group consisting of hypotaurine, thiotaurine, taurocholate.

In another particular embodiment, taurine derivatives are selected from different entities including the group consisting of acetylhomotaurinate, and piperidino-, benzamido-, phthalimido- or phenylsuccinylimido taurine derivatives. Such taurine derivatives are described notably by Kontro et al. (1983, Prog Clin Biol Res, Vol. 125 : 211-220) and by Andersen et al. (2006, Journal of pharmaceutical Sciences, Vol. 73(n° 1) : 106-108). Derivatives include for instance taurolidine (4,4′-methylene-bis(tetrahydro-2H-1,2,4-thiadiazine-1, 1-dioxide or taurolin), taurultam and taurinamide, chlorohydrate-N-isopropylamide-2-(1-phenylethyl) aminoethanesulfonic acid.

In another particular embodiment taurine analogs are selected from the group consisting of (+/−)piperidine-3-sulfonic acid (PSA), 2-aminoethylphosphonic acid (AEP), (+/−) 2-acetylaminocyclohexane sulfonic acid (ATAHS), 2-aminobenzenesulfonate (ANSA), hypotaurine,. ±trans-2-aminocyclopentanesulfonic acid (TAPS) 8-tétrahydroquinoléine sulfonic acid (THQS), N-2-hydroxyethylpiperazine-N′-2-ethane sulphonic acid (HEPES), beta-alanine, glycine, guanidinoethylsulfate (GES), 3-acétamido-1-propanesulfonic acid (acamprosate).

In another particular embodiment, substances required for taurine biosynthesis are selected from the group consisting of vitamin B6 (or pyridoxal-5′-phosphate), vitamin B12 (cobalamin), folic acid, riboflavin, pyridoxine, niacin, thiamine (thiamine pyrophosphate) and pantothenic acid.

An embodiment of the invention relates to a substance selected from the group consisting of taurine, a taurine precursor, a taurine metabolite, a taurine derivative, a taurine analog and a substance required for the taurine biosynthesis, for the prevention of brain intramyelinic oedema induced by an anti-convulsive drug.

Examples of anti-convulsive drugs that are susceptible to induce brain intramyelinic oedema include but are not limited to vigabatrin, carbamazepine, pregabalin, benzodiazepine, phenobarbital, ethosuximide, gabapentin, lamotrigine, phenytoin, valproate, topiramate, tiagabine and dilantin.

As used herein the term “vigabatrin” refers to 4-amino-5-hexenoic acid and encompasses the racemic mixture of vigabatrin or the active isomer.

An embodiment of the invention relates to a substance selected from the group consisting of taurine, a taurine precursor, a taurine metabolite, a taurine derivative, a taurine analog and a substance required for the taurine biosynthesis, for the prevention of brain intramyelinic oedema induced by vigabatrin.

This invention also relates to a therapeutic method for the prevention of brain oedema, wherein method comprises a step of administering to a subject in need thereof with an effective amount of a substance selected from the group consisting of taurine, a taurine precursor, a taurine metabolite, a taurine derivative, a taurine analog and a substance required for the taurine biosynthesis.

An embodiment of the invention relates to a therapeutic method for the prevention of brain intramyelinic oedema induced by an anti-convulsive drug such as above described, wherein method comprises a step of administering to a subject in need thereof with an effective amount of a substance selected from the group consisting of taurine, a taurine precursor, a taurine metabolite, a taurine derivative, a taurine analog and a substance required for the taurine biosynthesis.

An embodiment of the invention relates to a therapeutic method for the prevention of brain intramyelinic oedema induced by vigabatrin, wherein method comprises a step of administering to a subject in need thereof with an effective amount of a substance selected from the group consisting of taurine, a taurine precursor, a taurine metabolite, a taurine derivative, a taurine analog and a substance required for the taurine biosynthesis.

According to the invention, the term “subject” or “patient” and “subject in need thereof” or “patient in need thereof”, is intended for a human or a non-human mammal.

Generally speaking, a “therapeutically effective amount”, or “effective amount”, or “therapeutically effective”, as used herein, refers to that amount which provides a therapeutic effect for a given condition and administration regimen. This is a predetermined quantity of active material calculated to produce a desired therapeutic effect in association with the required additive and diluent; i.e., a carrier, or administration vehicle. Further, it is intended to mean an amount sufficient to reduce and most preferably prevent a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluents; i.e., carrier, or additive.

The present invention also relates to methods for the prevention or the treatment of convulsive disorders comprising a step of administering, to a subject in need thereof, a combination of (i) a first active ingredient which is an anti-convulsive drug; and (ii) a second active ingredient selected from the group consisting of taurine, a taurine precursor, a taurine metabolite, a taurine derivative, a taurine analog and a substance required for the taurine biosynthesis.

Examples of anti-convulsive drugs that are susceptible to induce brain intramyelinic oedema include but are not limited to vigabatrin, carbamazepine, pregabalin, benzodiazepine, phenobarbital, ethosuximide, gabapentin, lamotrigine, phenytoin, valproate, topiramate, tiagabine and dilantin.

The subjects in need of such treatments encompass those, either adult or child patients, which are susceptible to various convulsive disorders including primarily convulsive disorders. Convulsive disorders encompass epilepsy, tuberous sclerosis, infantile spasms as well as the convulsive disorders affecting patients undergoing a drug addiction, including a drug addiction to heroin or cocaine, ethanol.

The first and the second active ingredients are comprised in an anticonvulsive pharmaceutical composition in a “therapeutically effective amount”, that is in an amount sufficient for the combination of active ingredients to exert the expected anticonvulsive effect while inducing no brain intramyelinic oedema, or a brain intramyelinic oedema that is reduced as compared with pharmaceutical compositions comprising an anti-convulsive drug (e.g. vigabatrin) without taurine or a taurine-like substance.

### Pharmaceutical compositions

The present invention pertains to pharmaceutical compositions comprising a substance selected from the group consisting of taurine, a taurine precursor, a taurine metabolite, a taurine derivative, a taurine analog and a substance required for the taurine biosynthesis, for the prevention of brain oedema and more particularly brain intramyelinic oedema.

An embodiment of the invention also pertains to pharmaceutical compositions comprising a substance selected from the group consisting of taurine, a taurine precursor, a taurine metabolite, a taurine derivative, a taurine analog and a substance required for the taurine biosynthesis, for the prevention of brain intramyelinic oedema induced by an anti-convulsive drug such as above described.

An embodiment of the invention also pertains to pharmaceutical compositions comprising a substance selected from the group consisting of taurine, a taurine precursor, a taurine metabolite, a taurine derivative, a taurine analog and a substance required for the taurine biosynthesis, for the prevention of brain intramyelinic oedema induced by vigabatrin.

In particular, embodiment pharmaceutical compositions according to the invention comprise a first active ingredient which is an anti-convulsive drug (e.g. vigabatrin) and a second active ingredient which is taurine or a taurine-like substance.

Examples of anti-convulsive drugs that are susceptible to induce brain intramyelinic oedema include but are not limited to vigabatrin, carbamazepine, pregabalin, benzodiazepine, phenobarbital, ethosuximide, gabapentin, lamotrigine, phenytoin, valproate, topiramate, tiagabine and dilantin.

In particular, embodiment pharmaceutical compositions according to the invention comprise a first active ingredient which is vigabatrin and a second active active ingredient which is taurine or a taurine-like substance.

The pharmaceutical compositions according to the invention are suitable for treating various convulsive disorders including primarily convulsive disorders. Convulsive disorders encompass epilepsy, tuberous sclerosis, infantile spasms as well as the convulsive disorders affecting patients undergoing a drug addiction, including a drug addiction to heroin or cocaine, ethanol.

Thus, a pharmaceutical composition according to the invention consists primarily of an anti-convulsive pharmaceutical composition.

In a pharmaceutical composition according to the invention, the amount of the anti-convulsive drug (i.e. vigabatrin) is the amount of the said active ingredient that is conventionally used for treating patients affected with convulsive disorders. Typically, the said pharmaceutical composition is adapted so that the dosage form used allows the administration of an amount of said anti-convulsive drug (i.e. vigabatrin) ranging between 10 μg and 10 grams per day, preferably between 100 μg and 5 grams, including between 1 mg and 1 gram, for a human adult patient having a mean weight of 80 kilos. Typically, a dosage form will contain half the daily dose, for the purpose of performing two takes per day.

In a pharmaceutical composition according to the invention, the amount of the second active ingredient, i.e. taurine or a taurine-like substance, is adapted so that the said pharmaceutical composition is adapted so that the dosage form used allows the administration of an amount of taurine or of the taurine-like substance ranging from 10 μg to 10 grams per day for a human adult patient having a mean weight of 80 kilos.

Moreover in a pharmaceutical composition, the active ingredient(s) is (are) used in combination with one or more pharmaceutically or physiologically acceptable excipients.

Generally, a pharmaceutical composition according to the invention, irrespective of whether the said composition (i) comprises only one or more substances selected from taurine and taurine-like substances or (ii) comprises a combination of a first active ingredient which is the anti-convulsive drug (i.e. vigabatrin) and a second active ingredient selected from taurine and taurine-like substances, comprises the one or more active ingredients in an amount ranging from 0.1% to 99.9% by weight, and usually from 1% to 90% by weight, based on the total weight of the said pharmaceutical composition.

Generally, a pharmaceutical composition according to the invention comprises an amount of excipient(s) that ranges from 0.1% to 99.9% by weight, and usually from 10% to 99% by weight, based on the total weight of the said pharmaceutical composition.

By “physiologically acceptable excipient or carrier” is meant solid or liquid filler, diluents or substance which may be safely used in systemic or topical administration. Depending on the particular route of administration, a variety of pharmaceutically acceptable carriers well known in the art include solid or liquid fillers, diluents, hydrotropes, surface active agents, and encapsulating substances.

Pharmaceutically acceptable carriers for systemic administration that may be incorporated in the composition of the invention include sugar, starches, cellulose, vegetable oils, buffers, polyols and alginic acid. Specific pharmaceutically acceptable carriers are described in the following documents, all incorporated herein by reference: U.S. Pat. No. 4,401,663, Buckwalter et al. issued Aug. 30, 1983; European Patent Application No. 089710, LaHann et al. published Sep. 28, 1983; and European Patent Application No. 0068592, Buckwalter et al. published Jan. 5, 1983. Preferred carriers for parenteral administration include propylene glycol, pyrrolidone, ethyl oleate, aqueous ethanol, and combinations thereof.

Representative carriers include acacia, agar, alginates, hydroxyalkylcellulo se, hydroxypropyl methylcellulose, carboxymethylcellulose, carboxymethylcellulose sodium, carrageenan, powdered cellulose, guar gum, cholesterol, gelatin, gum agar, gum arabic, gum karaya, gum ghatti, locust bean gum, octoxynol 9, oleyl alcohol, pectin, poly(acrylic acid) and its homologs, polyethylene glycol, polyvinyl alcohol, polyacrylamide, sodium lauryl sulfate, poly(ethylene oxide), polyvinylpyrrolidone, glycol monostearate, propylene glycol monostearate, xanthan gum, tragacanth, sorbitan esters, stearyl alcohol, starch and its modifications. Suitable ranges vary from about 0.5% to about 1%.

For formulating a pharmaceutical composition according to the invention, the one skilled in the art will advantageously refer to the last edition of the European pharmacopoeia or of the United States pharmacopoeia.

Preferably, the one skilled in the art will refer to the fifth edition “2005” of the European Pharmacopoeia, or also to the edition USP 28-NF23 of the United States Pharmacopoeia.

The invention will be further illustrated by the following figures and examples. However, these examples and figures should not be interpreted in any way as limiting the scope of the present invention.

FIGURES

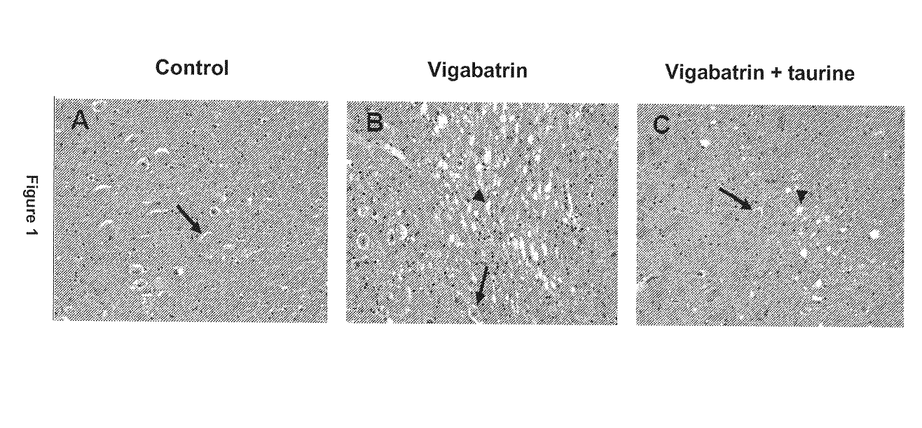

FIG. 1 illustrates the taurine effect on vigabatrin-induced brain vacuolation. Vacuolation in the brain tissue from vigabatrin-treated animals can be seen as small holes (arrowheads in B, C). This vacuolation is different from the fixation artifact found in nuclei (arrows in A, B, C). The vacuolation is extensive in vigabatrin-treated animals (B) whereas it is mild in vigabatrin-treated animals supplemented with taurine (C) and almost absent in control animals (A).

EXAMPLE:

Methods:

Animal treatments: As described previously (Duboc A. et al. 2004; Izumi Y. et al. 2004), Wistar rats Rj Wi IOPS Han were purchased from Janvier (Le Genest-St-Isle, France) at between six and seven weeks of age. vigabatrin dissolved in 0.9% NaC1 was administered at 40mg (125 mg/ml, 0.32m1) to rats by daily intraperitoneal injection for 65 days. These daily doses (rats: 200mg/kg) are in-line with those described for the treatment of epilepsy (adult patients: 1-6mg/kg; children: 50-75mg/kg; or infants: 100-150 mg/kg) (Chiron C. et al. 1997; Lux AL. et al. 2004; Aicardi J. et al. 1996). Taurine supplementation was given in drinking water at a concentration of 0.1M.

Histology: Rats were anesthetized by intraperitoneal injection (0.8 to 1.2 ml/kg) of a solution containing ketamine (40 mg/ml) and xylazine (4 mg/ml Rompum). Rat heads were removed and an opening created in the calvarium. These were then immersion fixed in 4% buffered formol (T/02170015, MMFrance) and then placed in phosphate buffered saline. Brains were then removed from the skulls and placed in 10% neutral buffered formalin. Brains were trimmed and their cerebellar nuclei placed in 10% buffered formalin and embedded in paraffin. Sections of the cerebellar nuclei were stained with hematoxylin and eosin.

Vacuolation of cerebellum was assessed by light microscopy, with the region of emphasis being the white matter adjacent to the cerebellar nuclei, and the nuclei. Vacuolation was scored as follows.

1- No or occasional individual vacuoles in perinuclear white matter.

2- Mild to moderate regional vacuolation of perinuclear white matter, sometimes extending into nucleus.

3- Extensive, widespread vacuolation of perinuclear white matter, often with involvement of adjacent nuclear regions. Vacuolation may extend to folial white matter

Results:

To investigate the role of taurine in vigabatrin-induced brain vacuolation, we supplemented the drinking water of a group of vigabatrin-treated rats with taurine (0.1M) (n=7) for 65. When the sections of the cerebellum were examined the white matter was found to contain a great number of vacuoles (FIG. 1B). These vacuoles were absent or very rare on the cerebellum of control animals (FIG. 1A). In animals supplemented with taurine, vacuoles were present in some animals but appeared at a much lower density (FIG. 1 C). When the vacuolation was scored in a blinded manner from a grade 1 (no or occasional) to the grade 3 (extensive), all control animals were scored 1 (n=6), all vigabatrin-treated animals were in grade 3 (n=7) and vigabatrin-treated animals with a taurine supplementation were either in grade 1 (n=3) or in grade 2 (n=4). This study clearly demonstrated that vigabatrin-induced brain vacuolation can be suppressed by taurine supplementation.

REFERENCES:

Aicardi J, Mumford JP, Dumas C, Wood S. Vigabatrin as initial therapy for infantile spasms: a European retrospective survey. Sabril IS Investigator and Peer Review Groups. Epilepsia. 1996;37:638-642

Anneken K, Evers S, Mohammadi S, Schwindt W, Deppe M. Transient lesion in the splenium related toantiepileptic drug: Case report and new pathophysiological insights. Seizure, 2008 in press.

Chason DP, Fleckenstein JL, Ginsburg MI, et al. Transient splenial edema in epilepsy:MR imaging evaluation. Proceedings of the 34th annual meeting of the American Society of Neuroradiology; Jun. 21-27 1996, Seattle, WA, USA. Chicago: Old Smith Printers, 1996.

Chiron C, Dumas C, Jambaque I et al. Randomized trial comparing vigabatrin and hydrocortisone in infantile spasms due to tuberous sclerosis. Epilepsy research. 1997;26:389-395

Duboc A, Hanoteau N, Simonutti M et al. Vigabatrin, the GABA-transaminase inhibitor, damages cone photoreceptors in rats. Annals of neurology. 2004;55:695-705

Kim SS, Chang KH, Kim ST, et al. Focal lesion in the splenium of the corpus callosum in epileptic patients: antiepileptic drug toxicity? Am J Neuroradiol 1999;20:125-9.

Lux AL, Edwards SW, Hancock E et al. The United Kingdom Infantile Spasms Study comparing vigabatrin with prednisolone or tetracosactide at 14 days: a multicentre, randomised controlled trial. Lancet. 2004;364:1773-1778.

Milh M, Villeneuve N, Chapon F, Pineau S, Lamoureux S, Livet MO, Bartoli C, Hugonenq C, Mancini J, Chabrol B, Girard N. Transient brain magnetic resonance imaging hyperintensity in basal ganglia and brain stem of epileptic infants treated with vigabatrin. J Child Neurol. 2009 Mar. ;24(3):305-15.

Pearl PL, Vezina LG, Saneto RP, McCarter R, Molloy-Wells E, Heffron A, Trzcinski S, McClintock WM, Conry JA, Elling NJ, Goodkin HP, de Menezes MS, Ferri R, Gilles E, Kadom N, Gaillard WD. Cerebral MRI abnormalities associated with vigabatrin therapy. Epilepsia. 2009 Feb. ;50(2):184-94. Epub 2008 Sep. 8.

Polster T, Hoppe M, Ebner A. Transient lesion in the splenium of the corpus callosum: three further cases in epileptic patients and a pathophysiological hypothesis. J Neurol Neurosurg Psychiatry 2001;70:459-463.

Wang QP, Jammoul F, Duboc A et al. Treatment of epilepsy: the GABA-transaminase inhibitor, vigabatrin, induces neuronal plasticity in the mouse retina. Eur J Neurosci. 2008;27:2177-2187.

Wheless JW, Carmant L, Bebin M, Conry JA, Chiron C, Elterman RD, Frost M, Paolicchi JM, Donald Shields W, Thiele EA, Zupanc ML, Collins SD. Magnetic resonance imaging abnormalities associated with vigabatrin in patients with epilepsy. Epilepsia. 2009 Feb. ;50(2):195-205. Epub 2008 Nov. 17.

1. A method for preventing brain oedema in a subject, comprising providing said subject with a substance selected from the group consisting of taurine, a taurine precursor, a taurine metabolite, a taurine derivative, a taurine analog, and a substance required for taurine biosynthesis.

2. The method of claim 1 wherein said brain oedema is associated with hypertensive encephalopathy, cerebral malaria, lesions of the anterior interosseous nerve, lateral amyotrophic sclerosis, Lyme disease, hepatic encephalopathy, Wernicke's encephalopathy, traumatic brain injury, stroke, intracerebral haemorrhage, an anti-convulsive drug, and intracranial hypertension.

3. A method for the prevention of brain oedema induced by an anti-convulsive drug, comprising the step of providing a subject that is or will be receiving said anti-convulsive drug which causes brain oedema a sufficient quantity of a substance selected from the group consisting of taurine, a taurine precursor, a taurine metabolite, a taurine derivative, a taurine analog, and a substance required for taurine biosynthesis to prevent brain oedema.

4. The method according to claim 3 wherein said anti-convulsive drug is selected from the group consisting of vigabatrin, pregabalin, benzodiazepine, phenobarbital, ethosuximide, gabapentin, lamotrigine, phenytoin, valproate, topiramate, tiagabine and dilantin.

5. The method according to claim 3 wherein said anti-convulsive drug is vigabatrin and said brain oedema is brain intramyelinic oedema.

6. The method according to claim 1, wherein said taurine precursor is selected from the group consisting of cysteine, cystathionine, homocysteine, S-adenosylhomocysteine, serine, N-acetyl-cysteine, glutathione, N-formylmethionine, S-adenosylmethionine, betaine and methionine.

7. The method according to claim 1 wherein said taurine metabolite is selected from the group consisting of hypotaurine, thiotaurine, and taurocholate.

8. The method substance according to claim 1, wherein said taurine derivative is selected from the group consisting of acetylhomotaurinate, and piperidino-, benzamido-, phthalimido- or phenylsuccinylimido taurine derivatives taurolidine, taurultam and taurinamide, chlorohydrate-N-isopropylamide-2-(1-phenylethyl) aminoethanesulfonic acid.

9. The method according to claim 1, wherein said taurine analog is selected from the group consisting of (+/−)piperidine-3-sulfonic acid (PSA), 2-aminoethylphosphonic acid (AEP), (+)2-acctylaminocyclohexane sulfonic acid (ATAHS), 2 - aminobenzenesulfonate (ANSA), hypotaurine, +trans-2-aminocyclopentanesulfonic acid (TAPS) 8-tetrahydroquinoleine sulfonic acid (THQS), N-2-hydroxyethylpiperazine-N-2-ethane sulphonic acid (HEPES), beta-alanine, glycine, guanidinoethylsulfate (GES), and 3-acetamido-l-propanesuIfonic acid (acamprosate).

10. The method substance according to claim 1, wherein the substance required for taurine biosynthesis is selected from the group consisting of vitamin B6, vitamin BI2, folic acid, riboflavin, pyridoxine, niacin, thiamine, and pantothenic acid.

11. A pharmaceutical composition for the prevention of brain oedema, comprising:
one or more anti-convulsive drugs; and
a substance selected from the group consisting of taurine, a taurine precursor, a taurine metabolite, a taurine derivative, a taurine analog, and a substance required for taurine biosynthesis to prevent brain intramyelinic oedema caused by said one or more anti-convulsive drugs.

12. The pharmaceutical composition of claim 11 further comprising one or more pharmaceutically acceptable excipients.

13. The pharmaceutical composition of claim 11 wherein said one or more anti-convulsive drugs are selected from the group consisting of vigabatrin, pregabalin, benzodiazepine, phenobarbital, ethosuximide, gabapentin, lamotrigine, phenytoin, valproate, topiramate, tiagabine and dilantin.

14. The pharmaceutical composition of claim 11 wherein said taurine precursor is selected from the group consisting of cysteine, cystathionine, homocysteine, S-adenosylhomocysteine, serine, N-acetyl-cysteine, glutathione, N-formylmethionine, S-adenosylmethionine, betaine and methionine.

15. The pharmaceutical composition according to claim 11 wherein said taurine metabolite is selected from the group consisting of hypotaurine, thiotaurine, and taurocholate.

16. The pharmaceutical composition according to claim 11 wherein said taurine derivative is selected from the group consisting of acetylhomotaurinate, and piperidino-, benzamido-, phthalimido- or phenylsuccinylimido taurine derivatives taurolidine, taurultam and taurinamide, chlorohydrate-N-isopropylamide-2-(1-phenylethyl) aminoethanesulfonic acid.

17. The pharmaceutical composition of claim 11 wherein said taurine analog is selected from the group consisting of (+/−)piperidine-3-sulfonic acid (PSA), 2-aminoethylphosphonic acid (AEP), (+)2-acctylaminocyclohexane sulfonic acid (ATAHS), 2- aminobenzenesulfonate (ANSA), hypotaurine, ±trans-2-arninocyclopentanesulfonic acid (TAPS) 8-tetrahydroquinoleine sulfonic acid (THQS), N-2-hydroxyethylpiperazine-N-2-ethane sulphonic acid (HEPES), beta-alanine, glycine, guanidinoethylsulfate (GES), and 3-acetamido-l-propanesulfonic acid (acamprosate).

18. The pharmaceutical composition according to claim 11 wherein the substance required for taurine biosynthesis is selected from the group consisting of vitamin B6, vitamin BI2, folic acid, riboflavin, pyridoxine, niacin, thiamine, and pantothenic acid.

19. The method according to claim 3 wherein said taurine precursor is selected from the group consisting of cysteine, cystathionine, homocysteine, S-adenosylhomocysteine, serine, N-acetyl-cysteine, glutathione, N-formylmethionine, S-adenosylmethionine, betaine and methionine.

20. The method according to claim 3 wherein said taurine metabolite is selected from the group consisting of hypotaurine, thiotaurine, and taurocholate.

21. The method according to claim 3 wherein said taurine derivative is selected from the group consisting of acetylhomotaurinate, and piperidino-, benzamido-, phthalimido- or phenylsuccinylimido taurine derivatives taurolidine, taurultam and taurinamide, chlorohydrate-N-isopropylamide-2-(1-phenylethyl)aminoethanesulfonic acid.

22. The method according to claim 3 wherein said taurine analog is selected from the group consisting of (+/−)piperidine-3-sulfonic acid (PSA), 2-aminoethylphosphonic acid (AEP), (+)2-acctylaminocyclohexane sulfonic acid (ATAHS), 2-aminobenzenesulfonate (ANSA), hypotaurine, +trans-2-aminocyclopentanesulfonic acid (TAPS) 8-tetrahydroquinoleine sulfonic acid (THQS), N-2-hydroxyethylpiperazine-N′-2-ethane sulphonic acid (HEPES), beta-alanine, glycine, guanidinoethylsulfate (GES), and 3-acetamido-l-propanesuIfonic acid (acamprosate).

23. The method according to claim 3 wherein the substance required for the taurine biosynthesis is selected from the group consisting of vitamin B6, vitamin BI2, folic acid, riboflavin, pyridoxine, niacin, thiamine, and pantothenic acid.
